(19) United States
(12) Patent Application Publication
(10) Pub. No.: US 2026/0216088 A1
(43) Pub. Date: Jul. 30, 2026

(54) IONIZABLE LIPIDS AND NANOPARTICLES COMPRISING SAME

(71) Applicant: MANA BIO LTD., Tel Aviv (IL)

(72) Inventors: Igor NUDELMAN, Haifa (IL); Maya KADURI, Haifa (IL); Lee GOLDFRYD, Nesher (IL); Galoz KANETI, Haifa (IL); Aviv ROTMAN, Tel Aviv (IL); Guy D. ROSIN, Tel Aviv (IL)

(21) Appl. No.: 19/146,586

(22) PCT Filed: Jan. 9, 2024
(86) PCT No.: PCT/IL2024/050033

§ 371 Date: Jul. 9, 2025

Related U.S. Application Data
(60) Provisional application No. 63/437,810, filed on Jan. 9, 2023.

(51) Int. Cl. A61K 9/51

(52) U.S. Cl. CPC A61K 9/5123; A61K 9/5146

(57) ABSTRACT

One or more ionizable lipid(s) and lipid nanoparticles comprising same and further encapsulating a polynucleic acid are provided. Pharmaceutical compositions comprising a therapeutically effective amount of the lipid nanoparticles encapsulating a therapeutically active polynucleic acid are also provided. Pharmaceutical compositions are used in delivering of the polynucleic acid to a lung tissue of a subject are also provided.

### CROSS REFERENCE TO RELATED APPLICATIONS

This application claims the benefit of priority under 35 USC § 119(e) of U.S. Provisional Patent Application No. 63/437,810, filed Jan. 9, 2023, entitled “IONIZABLE LIPIDS AND NANOPARTICLES COMPRISING SAME”, the content of which is incorporated herein by reference in its entirety.

### FIELD OF INVENTION

The present invention is directed to ionizable lipids and lipid nanoparticles comprising same and use thereof in pharmaceutical compositions.

### BACKGROUND OF THE INVENTION

New delivery methods for therapeutic and diagnostic compounds are in constant development. Although lipid-based nanoparticles are a well-known delivery modality, these agents are also constantly undergoing improvement. Among other concerns, the ability of a therapeutic carrier to effectively load and subsequently deliver the active agent to a target site is of great importance for reduced dosing, and improved treatment efficiency.

Although various ionizable lipids capable of encapsulation of hydrophilic agents such as DNA and/or RNA are known, there is a constant need for new and superior lipid nanoparticles (LNP). In particular, there is a great need for the development of new and superior LNP, which are capable of enhancing drug delivery to specific locations in the body. Specifically, there is an unmet need for lipid nanoparticles characterized by enhanced lung specificity and being suitable for administration of drugs specified to the lung.

### SUMMARY OF THE INVENTION

The present invention provides new compounds suitable for use as ionizable lipids. In addition, nanoparticles comprising same are provided. Compositions comprising the nanoparticles, which are useful for delivery of an active agent to a subject such as for treating or preventing a disease or disorder within the subject are also provided.

According to one aspect, A lipid nanoparticle comprising a compound, a salt of said compound or both, wherein the compound is represented by Formula 1:

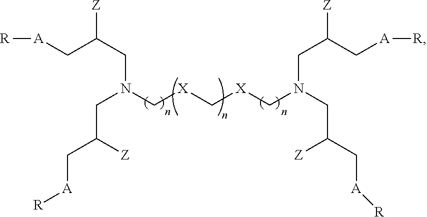

wherein: each X independently represents a —O—, —S—, CH2, or X is absent; each Z independently represents —OH or —SH; each A independently represents O or S; each n is independently between 0 and 5, and at least one n is not 0; each R independently is H, or comprises an optionally substituted C5-C30 alkyl; wherein said lipid nanoparticle further comprising an active agent, a helper lipid, a structural lipid and a modified lipid wherein: the helper lipid is a cationic lipid; a ratio of said compound relative to a total lipid content of said lipid nanoparticle is between 10 and 50 mol %; and a ratio of said structural lipid relative to said total lipid content of said lipid nanoparticle is between 5 and 50 mol %; and wherein said lipid nanoparticle is characterized by an average zeta potential in a range between −5 and +40 mV at a pH between 6 and 8.

In one embodiment, a molar ratio of said helper lipid and said modified lipid is between 1:0.2 and 1:0.01.

In one embodiment, a molar ratio of said compound and said helper lipid is between 0.2:1 and 5:1.

In one embodiment, a molar ratio of said structural lipid and said modified lipid is between 1:0.01 and 1:0.2.

In one embodiment, the active agent comprises a polynucleic acid; and a ratio between N:P within said LNP is between 1 and 20.

In one embodiment, a weight ratio between (i) the total amount of said compound, said helper lipid, said structural lipid and said modified lipid, and (ii) the polynucleic acid within said lipid nanoparticle is between 0.001:1 and 10:1

In one embodiment, a size distribution of said lipid nanoparticles is in a range between 50 and 500 nm; and wherein the cationic lipid is selected from DOTAP, DDAB, 18:1 EPC(1,2-dioleoyl-sn-glycero-3-ethylphosphocholine), and 18:0 EPC (1,2-distearoyl-sn-glycero-3-ethylphosphocholine), including any salt and any combination thereof.

In one embodiment, the lipid nanoparticle is characterized by any one of: an average zeta potential between 0 and +5 mV, at a pH between 6 and 8; the ratio between N:P is between 3 and 9; said structural lipid is cholesterol; said modified lipid is a PEG-lipid; a ratio of said helper lipid relative to said total lipid content of said lipid nanoparticle is between 35 and 45 mol %; and a ratio of the PEG-lipid relative to said total lipid content of said lipid nanoparticle is between 0.1 and 3 mol %.

In one embodiment, the lipid nanoparticles are characterized by pKa between 5 and 9; and wherein the compound comprises any one of MB-212 and MB 222:

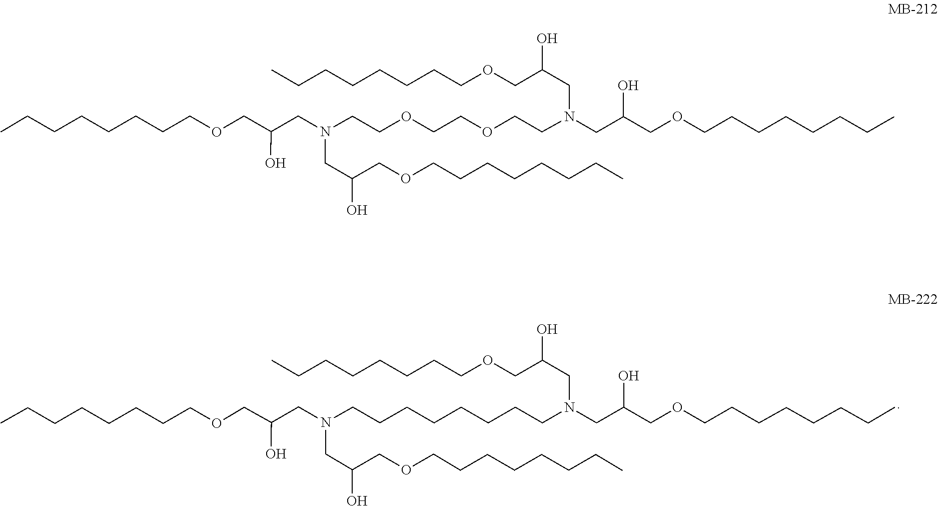

In one embodiment, upon administration to a subject, said LNP is characterized by at least 10 times higher expression within a lung, as compared to any one of liver, spleen and kidney of said subject.

In one embodiment, the ratio between N:P is about 5; wherein the cationic lipid is DOTAP; and wherein a ratio of said compound relative to a total lipid content of said lipid nanoparticle is between about 15 and about 25 mol %.

In another aspect, there is provided a pharmaceutical composition comprises a plurality of the lipid nanoparticles of the invention and a pharmaceutically acceptable carrier.

In one embodiment, the pharmaceutical composition comprises a therapeutically effective amount of the active agent.

In another aspect, there is provided a method for delivering an active agent to a lung tissue of a subject, the method comprising administering to said subject a therapeutically effective amount of the pharmaceutical composition of the invention, thereby delivering the active agent to said lung tissue.

In one embodiment, the therapeutically effective amount is between 0.01 and 5 mg/kg per day.

The method of the invention, wherein said administering is via intravenous, intratracheal, intranasal administration or via inhalation.

According to another aspect, there is provided a lipid nanoparticle comprising a compound, a salt of the compound or both, wherein the compound is represented by Formula I:

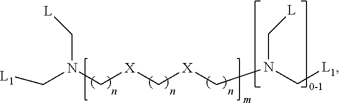

wherein: each L is independently R1,

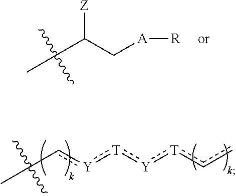

each L1 is independently R1,

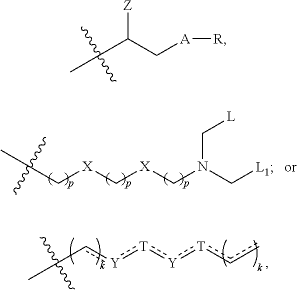

[symbol] represents a single bond, a triple bond or a double bond;
Z independently represents —OH or —SH;
A independently represents O or S;
each k is independently between 0 and 10; each Y independently is absent or comprises CH2, CHR′2, NR′2, NH, O, S, —CONH—, —CONR′—, —C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency;
each T independently represents an optionally substituted C5-C30 alkyl or an optionally substituted C5-C30 alkenyl;
each R′ is independently H or comprises an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl or a combination thereof,
each X independently represents a heteroatom, CH2, an optionally substituted C1-C10 alkyl, or X is absent;
each n and p is independently between 0 and 5, and at least one n is not 0;
m is between 1 and 3;
each R independently is H, or comprises an optionally substituted C5-C30 alkyl;
each R1 is an optionally substituted C1-C24 alkyl, and at least one L or L1 is or comprises,

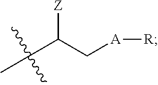

wherein the lipid nanoparticle further comprising an active agent, a helper lipid, a structural lipid and a modified lipid: wherein a ratio of the compound relative to a total lipid content of the lipid nanoparticle is between 15 and 55 mol %; wherein a ratio of the structural lipid relative to the total lipid content of the lipid nanoparticle is between 20 and 60 mol %.

In some embodiments, a molar ratio of the helper lipid and the modified lipid range between 1:0.2 and 1:0.01.

In some embodiments, a molar ratio of the compound and the helper lipid range between 0.5:1 and 5:1.

In some embodiments, a molar ratio of the structural lipid and the modified lipid range between 1:0.01 and 1:0.2.

In some embodiments, the active agent comprises a polynucleic acid.

In some embodiments, a weight ratio between (i) the total amount of the compound, the helper lipid, the structural lipid and the modified lipid, and (ii) the polynucleic acid within the lipid nanoparticle is between 0.001:1 and 10:1

In some embodiments, a size distribution of the lipid nanoparticles is in a range between 50 and 500 nm.

In some embodiments, the lipid nanoparticles are characterized by a zeta potential in a range between −5 and 40 mV, at a pH between 6 and 8.

In some embodiments, the lipid nanoparticles are characterized by pKa between 5 and 9.

In some embodiments, a ratio between N:P within the lipid nano particle ranges between 3 and 20.

In some embodiments, the ratio between N:P is about 12.

In another aspect, there is provided a pharmaceutical composition comprises a plurality of the lipid nanoparticles of the invention and a pharmaceutically acceptable carrier.

In some embodiments, the pharmaceutical composition comprising a therapeutically effective amount of the active agent.

In some embodiments, the pharmaceutical composition for use in the delivery of the active agent to a lung tissue.

In some embodiments, the pharmaceutical composition for use in the treatment of a lung disease, or a lung disorder.

In another aspect, there is provided a method for delivering an active agent to a lung tissue of a subject, the method comprising administering to the subject a therapeutically effective amount of the pharmaceutical composition of the invention, thereby delivering the active agent to the lung tissue.

In some embodiments, the therapeutically effective amount is between 0.01 and 1 mg/kg per day.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a bar graph showing in-vivo expression analysis of three LNP compositions: FMB-1143, FMB-748 and FMB-745. The bar graph presents a fluorescent signal intensity within the lung tissue, compared to heart tissue, spleen tissue, kidney tissue, and liver tissue.

### DETAILED DESCRIPTION OF THE INVENTION

The invention is based, at least in part, on a surprising finding that particular lipid nano particles (LNPs) of the invention showed lung specific activity in-vivo. Accordingly, the LNP of the invention can be used for specific delivery of an active agent to a lung tissue of a subject in need thereof.

Compounds disclosed by the present invention were discovered by methods of computational screening. Numerous LNP compositions were generated in-silico and ranked based on their predicted activity using a machine learning algorithm. After several optimization cycles in silico, a library containing several LNP compositions was obtained. Upon encapsulation of RNA into the LNP compositions, RNA activity was examined in-vivo. The disclosed LNP compositions were selected based on results obtained from in-vivo experiments, as exemplified hereinbelow.

### Lipid Nanoparticles

In one aspect of the invention, there is provided A lipid nanoparticle (LNP) comprising (i) a compound, including any salt thereof; (ii) a helper lipid, (iii) a structural lipid and (iv) a modified-lipid; wherein a molar ratio of the compound relative to a total lipid content of the composition is between about 15 and 55 mol %, or about 10 and 55 mol %, and wherein the compound is represented by Formula I:

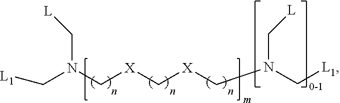

wherein: each L is independently R1,

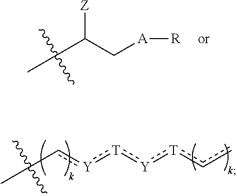

each L1 is independently R1,

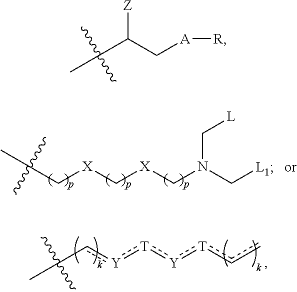

[symbol]

represents a single bond, a triple bond or a double bond; Z independently represents —OH or —SH; A independently represents O or S; each k is independently between 0 and 10; each Y independently is absent or comprises CH2, CHR′2, NR′2, NH, O, S, —CONH—, —CONR′—, —C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency; each T independently represents an optionally substituted C5-C30 alkyl or an optionally substituted C5-C30 alkenyl; each R′ is independently H or comprises an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl or a combination thereof; each X independently represents a heteroatom, CH2, an optionally substituted C1-C10 alkyl, or X is absent; each n and p is independently between 0 and 5, and at least one n is not 0; m is between 1 and 3; each R independently is H, or comprises an optionally substituted C1-C30 alkyl, or C5-C30 alkyl; each R1 is an optionally substituted C1-C24 alkyl, and at least one L or L1 is or comprises,

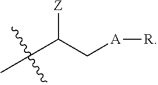

In some embodiments, there is provided a composition comprising a plurality of LNPs, wherein each LNP comprises (i) a compound, including any salt thereof; (ii) a helper lipid, (iii) a structural lipid and (iv) a modified-lipid; wherein a molar ratio of the compound relative to a total lipid content of the composition is between about 105 and 55 mol %, and wherein the compound is represented by Formula I:

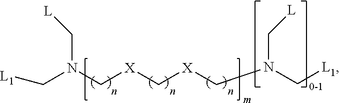

wherein: each L is independently R1,

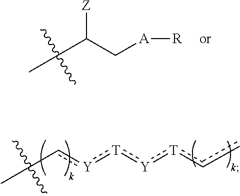

each L1 is independently R1,

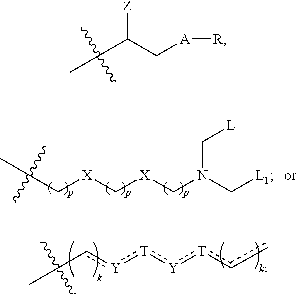

[symbol]

represents a single bond, a triple bond or a double bond; Z independently represents —OH or —SH; A independently represents O or S; each k is independently between 0 and 10; each Y independently is absent or comprises CH2, CHR′2, NR′2, NH, O, S, —CONH—, —CONR′—, C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency; each T independently represents an optionally substituted C5-C30 alkyl or an optionally substituted C5-C30 alkenyl; each R′ is independently H or comprises an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl or a combination thereof; each X independently represents a heteroatom, CH2, an optionally substituted C1-C10 alkyl, or X is absent; each n and p is independently between 0 and 5, and at least one n is not 0; m is between 1 and 3; each R independently is H, or comprises an optionally substituted C5-C30 alkyl, or C1-C30 alkyl; each R1 is an optionally substituted C1-C24 alkyl, and at least one L or L1 is or comprises,

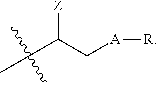

In some embodiments, the composition of the invention is an LNP composition (e.g. a solid composition comprising dried LNPs, or a liquid dispersion comprising LNPs dispersed or suspended within a solvent such as an aqueous solvent) comprising (i) a compound, including any salt thereof; (ii) a helper lipid, (iii) a structural lipid and (iv) a modified-lipid; wherein a molar ratio of the compound relative to a total lipid content of the composition is between about 15 and 55 mol %, or between about 10 and 55 mol %, and wherein the compound is represented by Formula I:

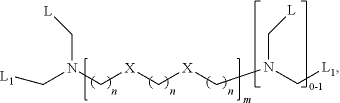

wherein: each L is independently R1,

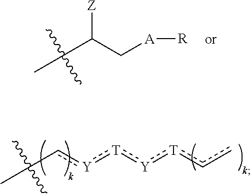

each L1 is independently R1,

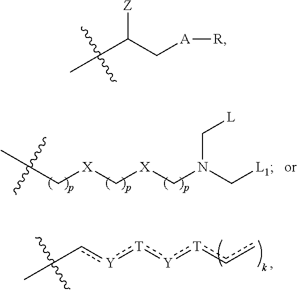

[symbol]

represents a single bond, a triple bond or a double bond; Z independently represents —OH or —SH; A independently represents O or S; each k is independently between 0 and 10; each Y independently is absent or comprises CH2, CHR′2, NR′2, NH, O, S, —CONH—, —CONR′—, C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency; each T independently represents an optionally substituted C5-C30 alkyl or an optionally substituted C5-C30 alkenyl; each R′ is independently H or comprises an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl or a combination thereof; each X independently represents a heteroatom, CH2, an optionally substituted C1-C10 alkyl, or X is absent; each n and p is independently between 0 and 5, and at least one n is not 0; m is between 1 and 3; each R independently is H, or comprises an optionally substituted C5-C30 alkyl or C1-C30 alkyl; and wherein each R1 is an optionally substituted C1-C24 alkyl, and at least one L or L1 is or comprises,

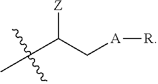

In some embodiments, the compound is represented by Formula II:

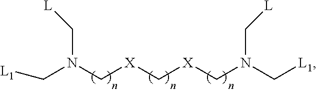

wherein each L and L1 is

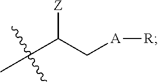

wherein X, Z, A and R are as describe above, and wherein each n is independently between 0 and 5, and at least one n is not 0. In some embodiments, the compound is represented by Formula II, wherein each X is selected from —O—, —S— and CH2; and wherein at least two R represent the optionally substituted C1-C30 alkyl.

In some embodiments, the compound is represented by Formula II or by Formula I, wherein a total number of C atoms of the R groups is between 5 and 50, between 10 and 50, between and 40, between 20 and 40, between 20 and 35, between 10 and 35, including any range between.

In some embodiments, the compound is represented by Formula II or by Formula I, wherein at least one R represents an optionally substituted alkyl being between 1 and 30, 5 and 30, 5 and 20, land 20, 1 and 10, 5 and 10 carbon atoms long, including any range between.

In some embodiments, the compound is represented by Formula II or by Formula I, wherein at least two R represent an optionally substituted alkyl being between 1 and 30, 5 and 30, 5 and 20, land 20, 1 and 10, 5 and 10 carbon atoms long, including any range between.

In some embodiments, the compound is represented by Formula II or by Formula I, wherein each R represents an optionally substituted alkyl being between 1 and 30, 5 and 30, 5 and 20, land 20, 1 and 10, 5 and 10 carbon atoms long, including any range between.

In some embodiments, the compound is represented by Formula 1:

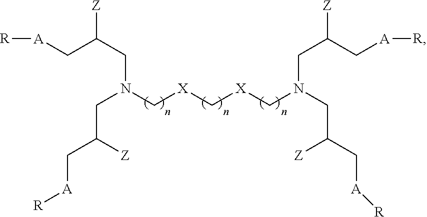

wherein Z, A, R and n are as described above; wherein each X independently represents a —O—, —S—, methylene, or X is absent; wherein at least one n is not 0. In some embodiments, the compound is represented by Formula 1, wherein at least one X is not absent, and wherein at least one R is not H. In some embodiments, the compound is represented by Formula 1, wherein at least two R are not H.

In some embodiments, the compound is represented by Formula 1, wherein a total number of C atoms of the R groups is between 10 and 60, between 5 and 50, between 5 and 60, between 510 and 40, between 20 and 40, between 20 and 35, between 10 and 35, including any range between. In some embodiments, the compound is represented by Formula 1, wherein at least one, or at least two R represent an optionally substituted alkyl being between 1 and 30, 5 and 30, 5 and 20, land 20, 1 and 10, 5 and 10 carbon atoms long, including any range between.

In some embodiments, the compound is represented by Formula 1, wherein each A is —O— and each Z is —OH; and wherein each R is an optionally substituted alkyl being between 1 and 30, 5 and 30, 5 and 20, land 20, 1 and 10, 5 and 10 carbon atoms long, including any range between.

In some embodiments, the compound is represented by Formula 2:

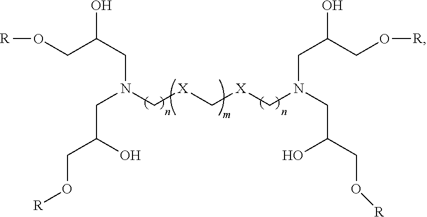

wherein X is as describe above, and each n is between 1 and 5, wherein m is between 0 and 3, and each R is an optionally substituted alkyl being between 1 and 30, 5 and 30, 5 and 20, land 20, 1 and 10, 5 and 10 carbon atoms long, including any range between. In some embodiments, the compound is represented by Formula 2, wherein each R is an optionally substituted C5-C10 alkyl.
In some embodiments, the compound comprises any one of MB-212 and MB 222, or both:

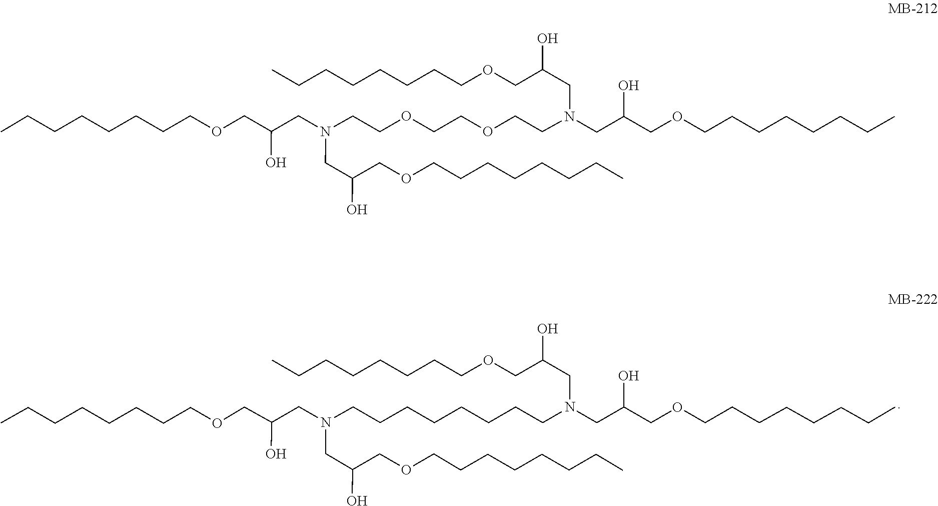

In some embodiments, each LNP comprises (i) the compound represented by Formula 1 or 2, including any salt thereof; (ii) the helper lipid, (iii) the structural lipid and (iv) the modified-lipid; wherein the helper lipid is a cationic lipid; wherein the structural lipid is a sterol; wherein the modified-lipid is a PEG lipid; and wherein the plurality of LNPs are characterized by a positive or neutral average zeta potential; and wherein each LNP encapsulates the active agent comprising a polynucleic acid.

In some embodiments, a molar concentration of one or more compounds of the invention within the LNP is between 10 and 55 mol %, 10 and 25, between 10 and 20, between 20 and 25, between, 15 and 30, between 20 and 35, between 30 and 40, between 30 and 35, between 20 and 55 mol %, including any range between. As used herein, the term “concentration” or “molar concentration” refers to a molar ratio relative to the total lipid content of the nanoparticle. A skilled artisan will appreciate that the molar ratios of the essential constituents (i.e., the compound of the invention, the helper lipid, the structural lipid, and the modified lipid) within the LNP and within the composition of the invention are identical. Thus, the molar concentrations and molar ratios disclosed herein for example with respect to LNP also encompass the corresponding molar concentrations and molar ratios within the composition of the invention and vice versa.

In some embodiments, the total lipid content refers to the combined content of the compound of the invention and of the structural lipid, the modified-lipid, and the helper lipid.

In some embodiments, a molar ratio of the structural lipid relative to the total lipid content of the composition is between 5 and 50 mol %, including any range or value in between. In some embodiments, a molar ratio of the compound (represented by any Formulae I-2) relative to a total lipid content of said lipid nanoparticle is between 10 and 55 mol %.

In some embodiments, a molar ratio of the compound of the invention relative to the total lipid content of the composition is between 10 and 55 mol %, between 15 and 25 mol %, between 15 and 20 mol %, between 20 and 25 mol %, between 15 and 30 mol %, between 20 and 35 mol %, between 30 and 40 mol %, between 30 and 35 mol %, between 20 and 55 mol %, between 20 and 55 mol %, including any range between. In some embodiments, a molar ratio of the compound of the invention relative to the total lipid content of the composition is between about 20 and about 40 mol %.

In some embodiments, a molar concentration of the helper lipid relative the total lipid content of the composition is between 5 and about 60 mol %, between 5 and 10 mol %, between 5 and 15 mol %, between 10 and 40 mol %, between 10 and 30 mol %, between 30 and 40 mol %, between 40 and 45, between 30 and 55 mol %, between 30 and 60 mol %, including any range between.

In some embodiments, the molar concentration of the helper lipid within the LNP is between about 35 and about 45, between about 38 and about 45 mol %, between about 38 and about 42 mol %, or about 40 mol % including any range between.

In some embodiments, a molar concentration of the structural lipid relative the total lipid content of the composition is between 5 and 50, between 15 and 60, between 15 and 50, between 20 and 25, between 20 and 30, between 20 and 40, between 25 and 45, between 35 and 40, between 35 and 45, between 35 and 50, between 40 and 50, between 45 and 50, between 35 and 50 mol %, including any range or value in between.

In some embodiments, a molar concentration of the structural lipid within the LNP is between 20 and 40 mol %, between 20 and 23 mol %, between 20 and 25 mol %, between 35 and 40 mol %, between 37 and 40 mol %, between 25 and 35 mol %, including any range between.

In some embodiments, a molar concentration of the modified lipid (e.g., PEG-lipid) within the LNP is between 0.5 and 10 mol %, between 0.1 and 10 mol %, between 0.1 and 0.5 mol %, between 0.5 and 1 mol %, between 1 and 5 mol %, between 0.5 and 2 mol %, between 5 and 10 mol %, between 5 and 7 mol %, between 7 and 10 mol %, including any range between.

In some embodiments, the LNP or the composition comprises a molar concentration of: (i) between about 20 and about 40% mol of the structural lipid and (ii) between about 1.5 and about 2.5% mol of the modified-lipid, and (iii) between about 35 and about 40% of the helper lipid and (iv) between about 20 and 35% mol of the compound of the invention.

In some embodiments, a weight ratio between the compound and the polynucleic acid within the LNP/composition is between 0.001:1 and 10:1, between 0.001:1 and 0.1:1, between 0.1:1 and 1:1, between 1:1 and 10:1, including any range between.

In some embodiments, a N:P ratio within the LNP or within the composition of the invention ranges between 1 and 20, between 3 and 6, between 3 and 9, between 6 and 8, between 6 and 8, between 8 and 10, between 10 and 12, between 6 and 12, between 6 and 13, and between 10 and 20, between 8 and 20, between 8 and 15, and between 4 and 5, including any range in between. The term “N:P ratio” refers to a ratio between N atoms of the compound of the invention and P atoms of the polynucleotide within the lipid nanoparticles or within the composition of the invention.

In some embodiments, the N:P ratio is about 5, or is selected from 3, 4, 5, and 6, or between 3 and 9, including any value or range between.

In some embodiments, the LNP consists essentially of the compound of the invention, the helper lipid, the structural lipid and the modified-lipid. In some embodiments, the LNP further encapsulates the active agent. In some embodiments, the LNP comprises a shell and an aqueous core, comprising the active agent. In some embodiments, the shell of the LNP comprises the compound of the invention, the helper lipid, the structural lipid and the modified lipid. In some embodiments, the active agent is incorporated within the shell, within the aqueous core or within the interface in between the shell and core. The term “shell” as used herein, refers to the outer portion of the particle, with a different composition than the core.

In some embodiments, the LNP consists essentially of the compound of Formula 1, the cationic helper lipid, sterol and the PEG-lipid; wherein the LNP further encapsulates the active agent; a molar ratio of the compound relative to a total lipid content of the composition is between about 15 and 40 mol %; a molar ratio of the cationic helper lipid relative to a total lipid content of the composition is between about 30 and 50 mol %, and wherein the LNPs within the composition are characterized by an average zeta potential in a range between −5 and +40 mV when measured at a pH between 6 and 8 (e.g. about 7).

In some embodiments, a combined molar concentration of the helper lipid and the compound of the invention within the LNP is between about 40 and about 75 mol %, or between about 60 and about 75 mol %; wherein a ratio between N:P is between 3 and 9; wherein an average particle size of the LNPs within the composition is between about 50 and 300 nm; and wherein the LNPs within the composition are characterized by an average zeta potential in a range between −5 and +40 mV, or between 0 and +10 mV when measured at a pH between 6 and 8 (e.g. about 7).

In some embodiments, the compound of the invention is of Formula 1 or 2; a combined molar concentration of the helper lipid and the compound of the invention within the LNP is between about 40 and about 75 mol % or between about 60 and about 75 mol %; a ratio of the helper lipid relative to the total lipid content of said lipid nanoparticle is between 35 and 45 mol %; a ratio of the PEG-lipid relative to the total lipid content of said lipid nanoparticle is between 1 and 5 mol %; wherein a ratio between N:P is between 8 and 12; wherein an average particle size of the LNPs within the composition is between about 50 and 300 nm; and wherein the LNPs within the composition are characterized by an average zeta potential in a range between −1 and +5 mV, when measured at a pH between 6 and 8 (e.g. about 7).

In some embodiments, the compound of the invention is of Formula 1 or 2; a combined molar concentration of the helper lipid and the compound of the invention within the LNP is between about 40 and about 75 mol %; a ratio of the helper lipid relative to the total lipid content of said lipid nanoparticle is between 35 and 45 mol %; a ratio of the PEG-lipid relative to the total lipid content of said lipid nanoparticle is between 1 and 5 mol %; wherein a ratio between N:P is about 12; wherein an average particle size of the LNPs within the composition is between about 50 and 300 nm; and wherein the LNPs within the composition are characterized by an average zeta potential in a range between 0 and +5 mV, when measured at a pH between 6 and 8 (e.g. about 7).

In some embodiments, the compound of the invention is of Formula 1 or 2; wherein the helper lipid is DOTAP; a combined molar concentration of the helper lipid and the compound of the invention within the LNP is between about 40 and about 75 mol %; a ratio of the helper lipid relative to the total lipid content of said lipid nanoparticle is between 30 and 50 mol %; a ratio of said compound relative to a total lipid content of said lipid nanoparticle is between about 10 and about 30 mol %; a ratio of the PEG-lipid relative to the total lipid content of said lipid nanoparticle is between 0.1 and 5 mol %; wherein a ratio between N:P is between about 4 and about 5; wherein an average particle size of the LNPs within the composition is between about 50 and 300 nm; and wherein the LNPs within the composition are characterized by an average zeta potential in a range between 0 and +5 mV, when measured at a pH between 6 and 8 (e.g. about 7).

In some embodiments, under suitable conditions at least one compound of the invention, the active agent, the helper lipid, the structural lipid and the modified-lipid spontaneously undergo self-assembly in an aqueous solution, so as to form the LNP. In some embodiments, the lipid nanoparticles are formulated to deliver one or more agents to one or more target cells.

In some embodiments, the LNP of the invention has a spherical geometry or shape. In some embodiments, the LNP has an inflated or a deflated shape. In some embodiments, a plurality of core-shell particles is devoid of any characteristic geometry or shape. In some embodiments, the LNP has a spherical shape, a quasi-spherical shape, a quasi-elliptical sphere, a deflated shape, a concave shape, an irregular shape, or any combination thereof.

In some embodiments, the plurality of core-shell particles within the composition of the invention are substantially spherically shaped, wherein substantially is as described herein. In some embodiments, the plurality of core-shell particles are substantially elliptically shaped, wherein substantially is as described herein. One skilled in the art will appreciate that the exact shape of each of the plurality of core-shell particles may differ from one particle to another. Moreover, the exact shape of the LNP may be derived from any of the geometric forms listed above, so that the shape of the particle does not perfectly fit a specific geometrical form. One skilled in the art will appreciate that the exact shape of the LNP may have substantial deviations (such as at least 5%, at least 10%, at least 20% deviation) from a specific geometrical shape (e.g., a sphere or an ellipse).

In some embodiments, a ratio between the compound relative to the total lipid content of the LNP of the invention is between 10 and 55%, between 15 and 25%, between 25 and 30%, between 25 and 35%, between 30 and 35%, between 35 and 45% and between 45 and 55%, including any range in between.

In some embodiments, a ratio between the structural lipid relative to the total lipid content of the LNP of the invention is between 5 and 50%, between 20 and 30%, between 25 and 30%, between 25 and 35%, between 30 and 35%, between 35 and 45%, between 45 and 50%, between 35 and 50%, including any range in between.

In some embodiments, a molar ratio of the helper lipid and the modified-lipid ranges between 1:0.2 And 1:0.01, between 1:0.15 and 1:0.01, between 1:0.1 and 1:0.01, between 1:0.05 and 1:0.01, including any range in between.

In some embodiments, a molar ratio of the compound and the helper lipid range between 1:0.5 and 1:5, between 1:0.5 and 4:1, between 1:0.5. and 3:1, between 1:0.5 and 2:1, between 1:0.5 and 1:1, between 1:0.1 and 5:1, between 1:0.25. and 5:1, between 1:0.5 and 2:1, between 1:1 and 1:2, between 1:1 and 1:5, including any range in between.

In some embodiments, a molar ratio of the structural lipid and the modified-lipid range between 200:1 and 2:1, between 100:1 and 5:1, between 100:1 and 10:1, between 100:1 and 30:1 between 100:1 and 50:1, between 100:1 and 70:1, between 100:1 and 90:1, between 100:1 and 100:3, between 100:1 and 20:1, including any range in between.

### Lipid Nanoparticle Composition

In some embodiments, the compound of the invention is represented by Formula I as described hereinabove. In some embodiments, the compound of the invention is an ionizable lipid. In some embodiments, the terms “compound”, “compound of the invention” and “ionizable lipid” are used herein interchangeably. In some embodiments, the heteroatom comprises O, N, NH, NR1, or S. In some embodiments, each X independently is O, or is absent.

In some embodiments, one of Rand R1 each independently represents a linear or a branched alkyl.

In some embodiments, L is

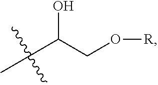

wherein R is as described herein. In some embodiments, each R represents the same or different alkyl.

In some embodiments, L is

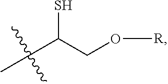

wherein R is as described herein. In some embodiments, each R represents the same or different alkyl.

In some embodiments, L is

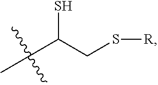

wherein R is as described herein. In some embodiments, each R represents the same or different alkyl.

In some embodiments, L is

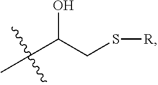

wherein R is as described herein. In some embodiments, each R represents the same or different alkyl.

As used herein the term “(C₃-C₁₀) cycloalkyl” is referred to an optionally substituted C3, C4, C5, C6, C7, C8, C9 or C10 ring. In some embodiments, (C₃-C₁₀) ring comprises optionally substituted cyclopropane, cyclobutene, cyclopentane, cyclohexane, or cycloheptane.

As used herein the term “C₃-C₁₀ heterocyclyl” is referred to an optionally substituted C3, C4, C5, C6, C7, C8, C9 or C10 heterocyclic aromatic and/or aliphatic, or unsaturated ring.

As used herein, the term “alkyl” describes an aliphatic hydrocarbon including straight chain and branched chain groups. In some embodiments, the alkyl group has 1 to 10 carbon atoms, 1 to 30 carbon atoms, or 5-30 carbon atoms. Whenever a numerical range e.g., “5-30”, is stated herein, it implies that the group, in this case the alkyl group, may contain 5 carbon atom, 6 carbon atoms, 10 carbon atoms, between 5 and 20, between 5 and 25, between 5 and 30, between 10 and 20, between 10 and 25, between 10 and 30, including any range between, up to and including 30 carbon atoms. The alkyl can be substituted or unsubstituted, as defined herein.

The term “alkyl”, as used herein, also encompasses saturated or unsaturated hydrocarbon, hence this term further encompasses alkenyl and alkynyl. In some embodiments, the alkyl group is a C1-C10 alkyl.

As used herein the term “C1-C10 alkyl” including any C1-C10 alkyl related compounds, is referred to any linear or branched alkyl chain comprising between 1 and 6, between 1 and 2, between 2 and 3, between 3 and 4, between 4 and 5, between 5 and 6, between 6 and 7, between 7 and 8, between 8 and 9, between 9 and 10 carbon atoms, including any range therebetween. In some embodiments, C1-C10 alkyl comprises any of methyl, ethyl, propyl, butyl, pentyl, iso-pentyl, hexyl, heptyl, octyl, nonyl, decyl and tert-butyl or any combination thereof. In some embodiments, C1-C10 alkyl as described herein further comprises an unsaturated bond, wherein the unsaturated bond is located at 1^{st}, 2^{nd}, 3^{rd}, 4^{th}, 5^{th}, 6^{th}, 7^{th}, 8^{th}, 9^{th} or 10^{th} position of the C1-C10 alkyl.

The term “alkenyl” describes an unsaturated alkyl, as defined herein, having between 2 and 30 carbon atoms and at least one carbon-carbon double bond. The alkenyl may be substituted or unsubstituted by one or more substituents, as described hereinabove.

The term “alkynyl”, as defined herein, is an unsaturated alkyl having between 2 and 30 carbon atoms and at least one carbon-carbon triple bond. The alkynyl may be substituted or unsubstituted by one or more substituents, as described hereinabove.

In some embodiments, the ionizable lipid is capable of undergoing ionization (protonation, or positive ionization) within a solution having a pH value below the pKa value of the ionizable lipid. In some embodiments, the ionizable lipid is capable of undergoing protonation within a solution having a pH value below the pKa value of the ionizable lipid. In some embodiments, at least 50 mol % of the ionizable moieties are positively charged (or protonated) within the composition of the invention having a pH value below the pKa value of the ionizable lipid.

In some embodiment, the compound of the invention has a MW of between 100 and 2000 Da, between 100 and 300 Da, between 100 and 500 Da, between 100 and 800 Da, between 300 and 500 Da, between 100 and 1,000 Da, between 500 and 800 Da, between 500 and 1,000 Da, between 800 and 1,200 Da, between 1000 and 2,000 Da, including any range between.

In some embodiments, the compound of the invention substantially comprises a single enantiomer of any one of the compounds described herein, wherein substantially is at least 70%, at least 75%, at least 80%, at least 85%, at least 90%, at least 92%, at least 93%, at least 95%, at least 97%, at least 98%, at least 99% by weight, including any value therebetween.

In some embodiments, the compound of the invention further encompasses any structurally similar functional derivative of the compounds disclosed herein, wherein structurally similar is at least 90%, at least 95%, at least 96%, at least 97%, at least 98%, at least 99% structure similarity, including any range between.

In some embodiments, the term “structure similarity” refers to a fingerprint similarity between two molecules. The term “fingerprint similarity” is well-understood by a skilled artisan. In some embodiments, the fingerprint similarity is calculated based on circular fingerprints, substructure keys-based fingerprints, and/or topological or path-based fingerprints.

Exemplary circular fingerprints include but are not limited to: Molprint 2D, ECFP (or Morgan fingerprint), FCFP, etc. In some embodiments, the term “structure similarity” as used herein, is calculated by Morgan fingerprint.

In some embodiments, a functional derivative refers to an ionizable lipid having a pKa value between 6.2 and 6.8, and capable of undergoing self-assembly in water so as to stably bind and/or encapsulate a polynucleic acid. In some embodiments, a functional derivative is further configured of cell internalizing a polynucleic acid (e.g., by forming a lipid nanoparticle as described herein). Cellular internalization can be determined as described hereinbelow.

In some embodiments, the compound is MB-212 or any salt thereof, as described hereinbelow.

In some embodiments, the compound is MB-222 or any salt thereof, as described hereinbelow.

In some embodiments, the active agent is a small molecule and/or a biologic molecule, such as polypeptide, a polynucleotide, etc. In some embodiments, the active agent is selected from a therapeutic agent, a prophylactic agent and a diagnostic agent including any combination thereof. In some embodiments, the one or more active agents are selected from the group consisting of: a protein, a peptide, a nucleic acid, a small molecule, and an antibody.

In some embodiments, the active agent is a lung therapeutic. In some embodiments, the active agent is an anticancer drug. In some embodiments, the active agent is an immunotherapy. In some embodiments, the active agent is an anti-infectious agent. In some embodiments, the active agent is an anti-inflammatory agent.

In some embodiments, the active agent is a polynucleic acid.

In some embodiments, the term “polynucleic acid” and the term “polynucleotide” are used herein interchangeably. In some embodiments, the polynucleotide comprises 60 to 15000 nucleobases, 15000 to 10000, 10000 to 4700, 200 to 5000 nucleobases, 300 to 5000 nucleobases, 400 to 5000 nucleobases, 400 to 2500 nucleobases, 200 to 3000 nucleobases, 400 to 2000 nucleobases, 400 to 1000 nucleobases, including any range between.

In some embodiments, the polynucleotide comprises at least 10 nucleobases, at least 250 nucleobases, at least 300 nucleobases, at least 350 nucleobases, at least 400 nucleobases, at least 450 nucleobases, at least 475 nucleobases, or at least 500 nucleobases. Each possibility represents a separate embodiment of the invention.

In some embodiments, the polynucleotide comprises 500 nucleobases at most, 750 nucleobases at most, 1,000 nucleobases at most, 1,250 nucleobases at most, 1,750 nucleobases at most, 2,500 nucleobases at most, 3000 nucleobases at most, 4000 nucleobases at most, or 5000 nucleobases at most. Each possibility represents a separate embodiment of the invention.

In some embodiments, the polynucleotide comprises a plurality of polynucleotide types. In some embodiments, the LNP comprises a plurality of polynucleotide types. In some embodiments, the composition comprises a plurality of nanoparticle types, each type of the LNP comprises a specific polynucleotide.

In some embodiments, a specific polynucleotide comprises a plurality of polynucleotide molecules harboring the same or an identical nucleic acid sequence. In some embodiments, a specific polynucleotide comprises a plurality of polynucleotide molecules harboring essentially the same nucleic acid sequence.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

As used herein, the term “polynucleotide types” refers to a plurality of polynucleotides each of which comprises a nucleic acid sequence differing from any one of the other polynucleotides of the plurality of polynucleotides by at least 1 nucleobase, at least 1 nucleobase, at least 1 nucleobase, at least 1 nucleobase, at least 1 nucleobase, or at least 10 nucleobases, or any value and range therebetween. Each possibility represents a separate embodiment of the invention.

In some embodiments, a polynucleotide comprises RNA, DNA, a synthetic analog of RNA, a synthetic analog of DNA, DNA/RNA hybrid, or any combination thereof. In some embodiments, the LNP of the invention comprises a polynucleotide selected from: RNA, DNA, a synthetic analog of RNA, a synthetic analog of DNA, DNA/RNA hybrid, or any combination thereof.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

The mRNA, as provided herein, comprises at least one (one or more) ribonucleic acid (RNA) polynucleotide having an open reading frame encoding at least one polypeptide of interest. In some embodiments, an RNA polynucleotide of an mRNA encodes 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5- 6, 6-10, 6-9, 6-8, 6-7, 7-10, 7-9, 7-8, 8-10, 8-9 or 9-10 polypeptides. In some embodiments, an RNA polynucleotide of an mRNA encodes at least 10, 20, 30, 40, 50, 60, 70, 80, 90 or 100 polypeptides. In some embodiments, an RNA polynucleotide of an mRNA encodes at least 100 or at least 200 polypeptides.

In some embodiments, the nucleic acids are therapeutic polynucleotide. As used herein, the term “therapeutic polynucleotide” refers to polynucleotide that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorder.

In some embodiments, therapeutic polynucleotide is an mRNA. In some embodiments, the mRNA is for treatment of lung disease. In some embodiments, the mRNA encodes a therapeutic protein to treat a lung disease. In some embodiments, the mRNA has a sequence Complementary to a mutant gene associated with the lung disease.

Thus, the polynucleotide of the invention can be used as therapeutic or prophylactic agents. They are provided for use in medicine. For example, the mRNA of the structures described herein can be administered to a subject, wherein the polynucleotides are translated in vivo to produce a therapeutic peptide.

In some embodiments, the polynucleotide comprises an inhibitory nucleic acid. In some embodiments, the polynucleotide comprises an antisense oligonucleotide.

As used herein, an “antisense oligonucleotide” refers to a nucleic acid sequence that is reversed and complementary to a DNA or RNA sequence.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments of the inhibitory nucleic acid has one or more chemical modifications to the backbone or side chains. In some embodiments, the inhibitory nucleic acid has at least one locked nucleotide, and/or has a phosphorothioate backbone.

Non-limiting examples of inhibitory nucleic acids useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro-RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable-either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein. In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense, and a combination thereof.

In some embodiments, the structural lipid is or comprises a sterol. In some embodiments, the term “structural lipid” encompasses a non-liposome forming lipid, as described herein. In some embodiments, the term “non-liposome forming lipid” is to be understood as referring to a lipid that does not spontaneously form into a vesicle when brought into an aqueous medium. There are various types of lipids that do not spontaneously vesiculate and yet are used or can be incorporated into vesicles. In some embodiments, the non-liposome forming lipid is or comprises a sterol.

In some embodiments, the structural lipid comprises any one of avenasterol, Betulin, Brassicasterol, Calcipotriol, campesterol, campestanol, cholesterol, cholesteryl hemisuccinate, cholesteryl sulfate, Daucosterol, DC-cholesterol, Dehydroergosterol, DMAPC-Chol, DMHAPC-Chol, ergosterol, Fucosterol, HAPC-Chol, Lupeol, MHAPC-Chol, OH—C-Chol, OH-Chol, Oleanolic acid, stigmastanol, stigmasterol, Ursolic acid, a hydrophobic vitamin (e.g. Vitamin D2, Vitamin D3, vitamin E, etc.), β-sitosterol, β-Sitosterol-Acetate, β-sitosterol-arginine, β-sitosterol-cysteine, β-sitosterol-glycine, β-sitosterol-histidine, β-sitosterol-serine, or a steroid, including any salt or any combination thereof.

In some embodiments, the structural lipid is cholesterol.

In some embodiments, the helper lipid is or comprises a phospholipid. In some embodiments, the helper lipid is or comprises a liposome forming lipid. As used herein, the term “liposome forming lipid” encompasses lipids (e.g., phospholipids) which upon dispersion or dissolution thereof in an aqueous solution at a temperature above a transition temperature (Tₘ), undergo self-assembly so as to form stable vesicles (e.g., lipid nanoparticles). As used herein, the term Tm refers to a temperature at which the lipids undergo phase transition from solid (ordered phase, also termed as a gel phase) to a fluid (disordered phase, also termed as fluid crystalline phase). Tm also refers to a temperature (or to a temperature range) at which the maximal change in heat capacity occurs during the phase transition.

In some embodiments, the phospholipid encompasses a single phospholipid specie or a plurality of chemically distinct phospholipids.

In some embodiments, the liposome forming lipid is a phospholipid having one or two C12 to C24 hydrocarbon tails, typically, acyl, alkyl or alkenyl chain) and have varying degrees of unsaturation, from being fully saturated to being fully, partially or non-hydrogenated lipids (the level of saturation may affect rigidity of the liposome thus formed (typically liposomes formed from lipids with saturated chains are more rigid than liposomes formed from lipids of same chain length in which there are un-saturated chains, especially having cis double bonds). In some embodiments, at least one of the liposome forming lipid is a phospholipid having one or two C12 to C20, C16 to C20, or C16 to C18 hydrocarbon tails, including any value and range therebetween. In some embodiments, the liposome forming lipid is fully saturated, linear, or branched.

Further, the phospholipid may be of natural source (e.g., naturally occurring phospholipids), semi-synthetic or fully synthetic lipid, as well as electrically neutral (e.g., zwitterionic), negatively, or positively charged.

Non-limiting examples of neutral phospholipids include but are not limited to diacylphosphatidylcholines, dialkylphosphatidylcholines, sphingomyelins, and diacylphosphatidylethanolamines. Phosphatidylcholines (PC), including those obtained from egg, soybeans or other plant sources or those that are partially or wholly synthetic, or of variable lipid chain length and unsaturation are suitable for use in the present compositions. Synthetic, semisynthetic and natural product phosphatidylcholines including, but not limited to, POPC, DOPC, DMPC, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), and dipalmitoylphosphatidylcholine (DPPC) are suitable phosphatidylcholines for use in the preparation of liposomes. Charged phospholipids can include phosphatidylglycerols, cardiolipins, or headgroup modified lipids such as N-succinyl-phosphatidylethanolamines, N-glutaryl-phosphatidylethanolamines, and PEG-derivatized phosphatidylethanolamines.

In some embodiments, the helper lipid is a cationic lipid. In some embodiments, the helper lipid is devoid of non-cationic lipids.

In some embodiments, the cationic lipid comprises at least one permanent positive charge (i.e. at least one non-ionizable cationic moiety). In some embodiments, the cationic lipid is a tri-alkyl ammonium-based lipid. In some embodiments, the cationic lipid comprises between 1 and 5, or between 1 and 3 positive charges.

Non-limiting examples of cationic lipids include but are not limited to 5-carboxyspermylglycinedioctadecylamide or “DOGS,” N-[1-(2,3-dioleyloxy)propyl]-N,N,N-trimethylammonium chloride or “DOTMA”, 2,3-dioleyloxy-N-[2(spermine-carboxamido)ethyl]-N,N-dimethyl-1-pr-opanaminium or “DOSPA”, 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-oc-tadecadienoxy)propane or “CLinDMA”, 2-[5′-(cholest-5-en-3-beta-oxy)-3′-oxapentoxy)-3-dimethy 1-1-(cis,cis-9′,1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-di-methylethanamine (DLin-KC2-DMA)).

In some embodiments, the helper lipid is or comprises a non-cationic lipid. As used herein, the term “non-cationic lipid” refers to any neutral, or zwitterionic lipid. Non-cationic lipids include, but are not limited to, dioleoylphosphatidylethanolamine (DOPE), distearoylphosphatidylcholine (DSPC), hydrogenated soy phosphatidylcholine (HSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), or a mixture thereof.

In some embodiments, the helper lipid is a cationic lipid. In some embodiments, the cationic lipid is or comprises any of DOTAP, DDAB, 1,2-dioleoyl-sn-glycero-3-ethylphosphocholine, 18:1 EPC(1,2-dioleoyl-sn-glycero-3-ethylphosphocholine), and 18:0 EPC (1,2-distearoyl-sn-glycero-3-ethylphosphocholine), or any combination thereof.

In some embodiments, the modified-lipid is a PEG-modified lipid. In some embodiments, the modified-lipid comprises a single PEG moiety covalently bound to the head group of the lipid. In some embodiments, the modified-lipid comprises a plurality of PEG moieties covalently bound to the head group of the lipid. In some embodiments, the PEG moiety comprises an alkylated PEG such as methoxy poly (ethylene glycol) (mPEG). The PEG moiety can have a molecular weight of the head group from about 750 Da to about 10,000 Da, at times, from about 750 Da to about 6,000 Da and typically between about 1,000 Da to about 5,000 Da, or about 2000 Da, including any range between.

In some embodiments, the modified-lipid is or comprises DMG-PEG2000.

### Lipid Nanoparticle Properties

In some embodiments, the pKa value of LNP of the invention is between 5 and 9, including any range between. In some embodiments, the pKa value of the LNP is between 5 and 8, between 5 and 6, between 6 and 8, between 6 and 7, between 7 and 9, including any range between.

In some embodiments, the pKa value of the LNP of the invention is between 6 and 7, between 6 and 6.2, between 6.2 and 6.4, between 6.4 and 6.6, between 6.6 and 6.8, and between 6.8 and 7, including any range in between.

In some embodiment, the LNP is characterized by an average particle size of less than 500 nm to facilitate its entrance through the extracellular matrix to a cell. In one embodiment, the carrier is characterized by an average particle size of less than 300 nm in diameter to facilitate its entrance through the extracellular matrix to a cell.

In some embodiment, the LNP is characterized by an average particle size of less than 500 nm, less than 400 nm, less than 300 nm, less than 350 nm, less than 200 nm, less than 100 nm, less than 50 nm, including any range between.

In some embodiment, the LNP is characterized by an average particle size of between 50 and 500, between 50 and 250 nm, between 50 and 400 nm, between 100 and 300 nm, between 200 and 400 nm, between 250 and 350 nm, including any range between.

In some embodiment, the LNP is characterized by a positive zeta potential (when measured at a pH about 7, e.g., between about 6.5 and 7.5). In some embodiments, the LNP is characterized by zeta potential ranging between −5 mV and +40 mV, including any range between. In some embodiments, the LNP is characterized by zeta potential ranging between −3 mV and +20 mV, between −3 mV and +10 mV, between −2 mV and +10 mV, between −1 mV and +10 mV, between 0 mV and +20 mV, between −0 mV and +10 mV, between −0 mV and +5 mV, including any range between when measured at physiological pH (such as a pH between 6.5 and 7.5 including any range between).

In some embodiments, the LNP is stable for a time period ranging between 1 day and 1 year, or more, including any range between. In some embodiments, the term “stable” refers to physical and chemical stability of the dry nanoparticle (such as being substantially devoid of phase separation, agglomeration, disintegration, and/or substantially retaining the initial loading of the active agent) under appropriate storage conditions. In some embodiments, the term “stable” refers to physical and chemical stability of the nanoparticle within an aqueous solution or within the composition of the invention (e.g., dispersion stability).

The inventors have surprisingly found that upon administration to a subject (i.e. animal subject), the LNP composition of the invention is characterized by a distinct activity profile in vivo, compared to similar LNP compositions devoid of structural lipid and/or helper lipid, or having a different ratio between the LNP constituents. As demonstrated in the Examples section, the LNPs of the invention are characterized by enhanced specificity and consequently in enhanced activity within the lung tissue, as compared to other organs (such as kidney, liver, heart, etc.) of the subject.

In some embodiments, the LNP of the invention is characterized by enhanced specificity to the lung (e.g., lung tissue, lung cell) of a subject. In some embodiments, the LNP of the invention is characterized by enhanced specificity to the lung compared to a similar LNP comprising, a commercialized ionizable lipid (Dlin-MC3-DMA). The term “enhanced specificity” as used herein encompasses the property of the LNP to undergo specific expression in the lungs of the subject, as compared to other organs (such as kidney, liver, heart, etc.). In some embodiments, enhanced specificity; comprises at least 5, at least 10, at least 50, at least 100, at least 250, at least 500, at least 1000 times greater expression of the mRNA within the lung, as compared to another organ (such as kidney, spleen, liver, or heart). In some embodiments, upon administration to a subject, the LNP of the invention is characterized by at least 10 times, at least 50, at least 100, at least 250, at least 500, at least 1000 times or between 10 and 500, between 10- and 200-times higher expression within a lung, as compared to any one of liver, spleen and kidney of the subject, including any range between.

The specificity of the LNP can be determined for example by performing a tissue expression analysis in-vivo (e.g., by utilizing LNPs encapsulating a signal emitting probe or using an appropriate imaging technique such as luminescence).

### Pharmaceutical Composition

In another aspect, the LNPs of the invention can be used as a therapeutic or prophylactic agents. In some embodiments, the LNPs are in a composition. In some embodiments, the composition is a pharmaceutical composition. In some embodiments, the pharmaceutical composition comprises a plurality of the LNPs and a pharmaceutically acceptable carrier, wherein the LNPs are the same or different. In some embodiments, “different LNPs” refer to lipid nanoparticles with different active agent. In some embodiments, the pharmaceutically acceptable carrier of the active agent is the LNPs of the invention.

In some embodiments, the pharmaceutically acceptable carrier is also referred to as an excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Hartmann solution, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

In some embodiments, the LNP of the invention comprising the compound of the invention, structural lipid, helper lipid and modified lipid as well as in a relative molar ratio as described in the embodiments herein above, to the compound of the invention is based on the active agent to be delivered as well as its target. In some embodiments, the relative molar ratio as described in the embodiments herein above, to the compound of the invention enables specific targeting of the LNPs to the lung.

The carrier may comprise, in total, from about 0.1% to about 99.99999% by weight of the pharmaceutical compositions presented herein.

In some embodiments, the pharmaceutical composition comprising the LNPs of the invention further comprises an effective amount of the active agent described herein. In some embodiments, the pharmaceutical composition comprises a therapeutically effective amount of the active agent described herein. In some embodiments, the therapeutically effective amount is sufficient for reduction of at least one symptom, or for substantial reduction in the severity and/or inhibition of the progression of a disease, disorder, or condition as described hereinabove.

In some embodiments, the pharmaceutical composition is for use in the treatment or prevention of a lung disease. In some embodiments, the lung disease is selected form lung cancer, ARDS, lung fibrosis, viral infection, bacterial infection, COPD, asthma, bronchiectasis, bronchiolitis, bronchitis, cystic fibrosis, emphysema, mesothelioma, pleural effusion, pleurisy, pneumonia, pneumothorax, RSV, SARS, SARS-CoV-2, silicosis, tuberculosis, whooping cough and influenza. Lung cancers are well known in the art and include for example, small cell lung cancer (SCLC), non-small cell lung cancer (NSCLC), lung adenocarcinoma, lung squamous cell carcinoma, lung large cell carcinoma, Pancoast tumors and carcinoid tumors. The term “effective amount” or “therapeutically effective amount” refers to an amount effective, at a dosages and periods of time necessary to achieve a desired therapeutic result. It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. In some embodiments, the therapeutically effective amount of the active agent administered daily to the subject is reduced, as compared to IV administration of a pharmaceutical composition comprising the same active agent which is not encapsulated within the LNP of the invention. In some embodiments, the therapeutically effective amount of the active agent administered daily to the subject is reduced, as compared to a similar pharmaceutical composition comprising the same active agent encapsulated within a commercial LNP (e.g., Dlin-MC3-DMA). In some embodiments, reduced therapeutically effective amount is by at least 2 times, at least 5 times, at least 10 times, at least 20 times, at least 50 times, at least 100 times, including any range between.

In some embodiments, the LNPs of the invention are composed of pharmaceutically acceptable ingredients (such as the phospholipids lipids and sterol) or pharmaceutically acceptable salt thereof. In some embodiments, the pharmaceutical composition comprises the LNPs of the invention, a pharmaceutically acceptable salt thereof or both.

In some embodiments, the pharmaceutical composition materials are characterized by a pharmaceutical grade. In some embodiments, the pharmaceutical grade are characterized by a chemical purity of at least 90%, at least 95%, at least 99%, including any range in between.

The term “pharmaceutically acceptable” can mean approved by a regulatory agency of the Federal or a state government or listed in the U. S. Pharmacopeia or other generally recognized pharmacopeia for use in animals, and more particularly in humans.

In some embodiments, the pharmaceutical composition of the invention is for use as a medicament. In some embodiments, the pharmaceutical composition is formulated for administration to a lung, lung tissue or lung cell. In some embodiments, the pharmaceutical composition is formulated for systemic administration. In some embodiments, the pharmaceutical composition is formulated for intravenous (IV) administration. In some embodiments, pharmaceutically acceptable carrier comprises a buffer.

In some embodiments, the administration to the lung (e.g. tissue, cell) is by IV. In some embodiments, the administration to the lung (e.g. tissue, cell) is systemic.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, intravenous, subcutaneous, oral, intramuscular, intrathecal, inhaled, intracerebroventricular, intravitreal, transdermal, or intraperitoneal. In some embodiments, the pharmaceutical composition is for use in a therapeutic method. In some embodiments, a therapeutic method is a method of treatment. In some embodiments, the pharmaceutical composition is for use in treating a medical condition. In some embodiments, the medical condition is a condition, a disease, or a disorder. In some embodiments, the pharmaceutical composition is for use in a diagnostic method.

In some embodiments, the pharmaceutical composition is formulated for administration to a subject. In some embodiments, the method comprises administering the pharmaceutical composition of the invention to a subject. In some embodiments, the subject is a mammal. In some embodiments, the mammal is a human. In some embodiments, the subject is in need of pharmaceutical composition. In some embodiments, the pharmaceutical composition is for use in treatment or prevention of a disease or condition in humans and other mammals. In some embodiments, the subject is in need of treatment. In some embodiments, the subject is a volunteer for a diagnostic method. In some embodiments, the subject is in need of diagnosis.

The active therapeutic agents of the invention include the lipid nanoparticles, or protein translated from the polynucleotides contained in the lipid nanoparticles.

In some embodiments, the mammal is a laboratory animal. Examples of laboratory animals include, but are not limited to, mice, rats, rabbits, hamsters, dogs, pigs, and monkeys. In some embodiments, the mammal is a mouse or rat.

In some embodiments, the pharmaceutical composition of the invention is in a form of a solutions, a suspension, an emulsion, a tablet, a pill, a capsule, a powder, a gel, a cream, an ointment, a foam, a paste, a sustained-release formulations and the like. In some embodiments, the pharmaceutical compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc.

### Method for Use

According to another aspect, there is provided a method for delivering an active agent to a lung, a lung tissue or a lung cell of a subject, the method comprising administering to the subject a therapeutically effective amount of the pharmaceutical composition of the invention described hereinabove, thereby delivering the active agent to the lung. In some embodiment, the pharmaceutical composition is for use in delivery of the active agent to the lung (e.g., lung tissue, lung cell) of the subject.

In some embodiments, the pharmaceutical composition is for use in the treatment of a lung disease, a lung disorder, or a lung condition. In some embodiments, pharmaceutical composition comprises a therapeutically effective amount of the active agent.

In some embodiments, the pharmaceutical composition is a lung targeting composition. In some embodiments, the pharmaceutical composition is for use in treating a lung disease. In some embodiments, the lung disease comprises lung inflammation. Examples of lung diseases comprise, but are not limited to: lung cancer, lung fibrosis, viral infection, bacterial infection, COPD, asthma, bronchiectasis, bronchiolitis, bronchitis, cystic fibrosis, emphysema, mesothelioma, pleural effusion, pleurisy, pneumonia, pneumothorax, RSV, SARS, SARS-CoV-2, silicosis, tuberculosis, whooping cough and influenza. Lung cancers are well known in the art and include for example, small cell lung cancer (SCLC), non-small cell lung cancer (NSCLC), lung adenocarcinoma, lung squamous cell carcinoma, lung large cell carcinoma, Pancoast tumors and carcinoid tumors.

In some embodiments, the method comprises administering an effective amount of the pharmaceutical composition. In some embodiments, an effective amount is the human equivalent of a murine dose of between 0.01 and 5 mg/kg body weight per day. In some embodiments, the human equivalent of the murine dose depends upon the route of administration. In some embodiments, an effective amount is the human equivalent of a murine dose of between 0.01 and 5, between 0.1 and 5, between 0.1 and 2, between 0.01 and 2, between 0.01 and 3, between 0.01 and 1, between 0.5 and 5 mg/kg body weight, including any value in between.

The dosage administered will be dependent upon the age, health, and weight of the recipient, kind of concurrent treatment, if any, frequency of treatment, and the nature of the effect desired.

### Definitions

As used herein, the term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “substantially” refers to at least 85%, at least 90%, at least 92%, at least 95%, at least 96%, at least 97%, at least 98%, at least 99%, or between 60 and 99.9%, between 70 and 80%, between 70 and 90%, between 80 and 90%, between 90 and 95%, between 95 and 99.9%, including any range or value therebetween.

As used herein, the term “substituent” encompasses hydrogen, halogen, —NO₂, —CN, —OH, oxo, imino, —CONH₂, —CONR′₂, —CNNR′₂, —CSNR′₂, —CONH—OH, —CONH—NH₂, —NHCOR, —NHCSR, —NHCNR, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl —SR′, or a combination thereof.

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N) NR″— linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

As used herein, the term “substituted” or the term “substituent” are related to one or more (e.g., 2, 3, 4, 5, or 6) substituents, wherein the substituent(s) is as described herein.

### General

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

Exemplary compounds of the invention (MB-212 and MB-222, depicted hereinbelow) have been synthesized according to synthetic schemes presented hereinbelow. The inventors successfully formed the lipid nanoparticles of the invention using MB-212 or MB-222 and tested their ability to deliver an active agent to the lung in-vivo. Exemplary compounds of the invention (ionizable lipids) are presented hereinbelow.

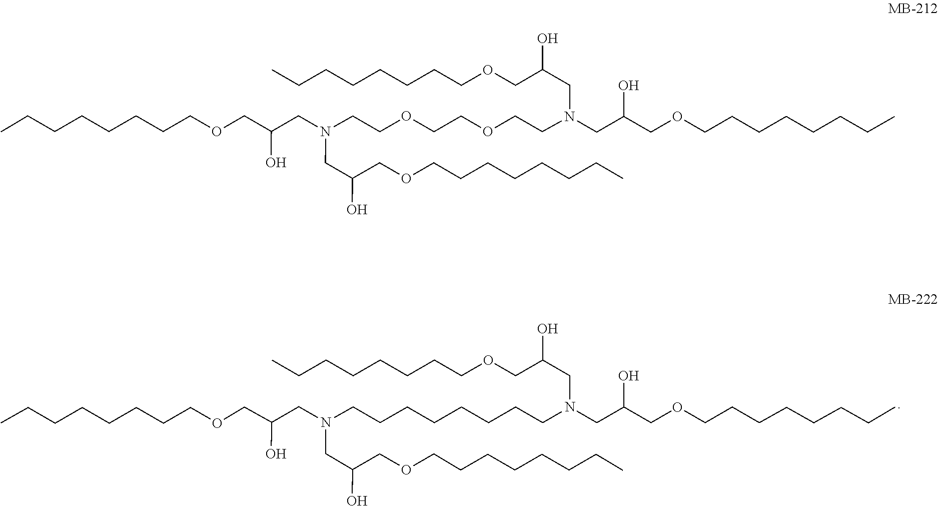

A general synthetic scheme for some of the exemplary compounds of the invention is presented herein. Other possible synthetic strategies are well-known to a skilled artisan.

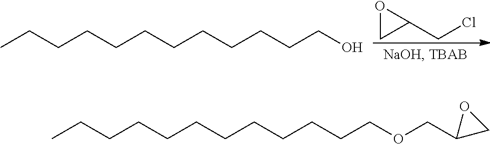

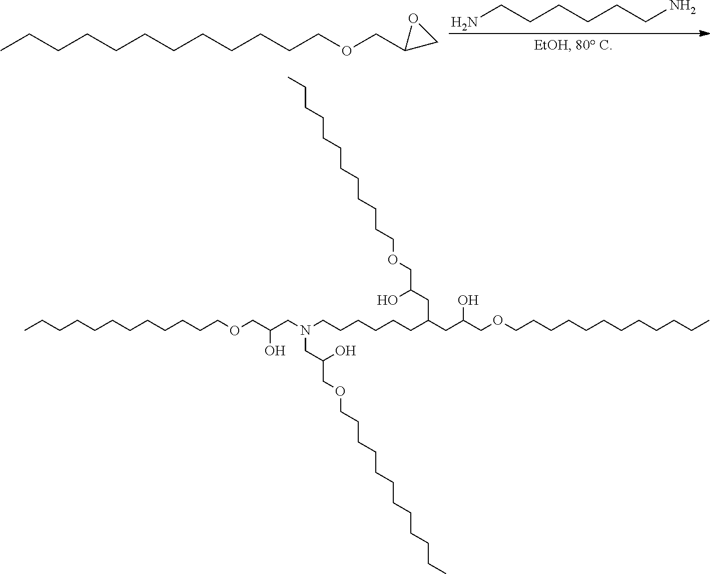

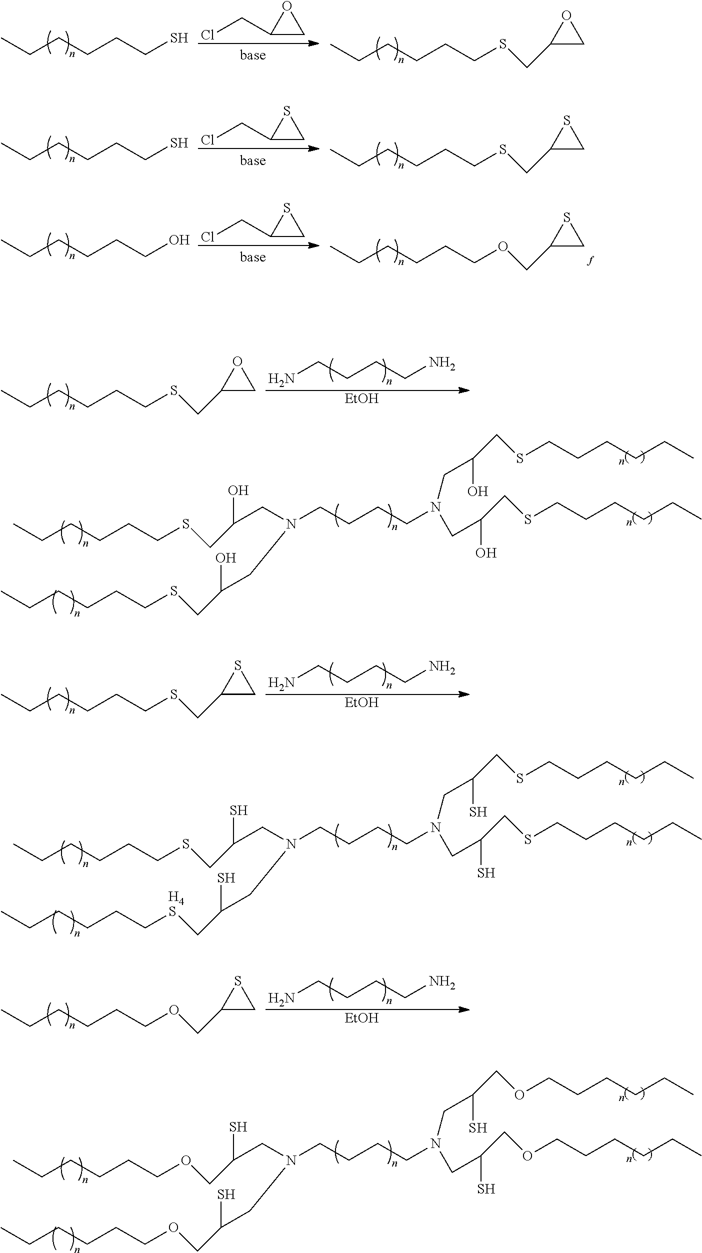

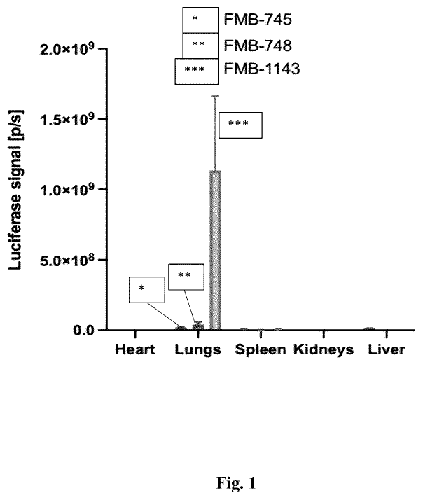

Some of the exemplary compositions of the invention have been characterized by enhanced specificity to lung cells, as determined in-vivo in mice studies. For example, LNPs of the invention contain MB-212 or MB-222 as the ionizable lipid, exhibited enhanced specificity to the lung tissue, as compared to a similar composition comprising Dlin-MC3-DMA as the ionizable lipid (FIG. 1). Surprisingly, the inventors found that exemplary LNPs containing between about 30 and about 50 of a helper lipid (e.g., DOTAP), between about 20 and about 50 of a structural lipid (cholesterol), and between about 0.1 and about 5 of a modified lipid (e.g., PEG-lipid), along with between about 15 and about 50% of the ionizable lipid (e.g., MB-212 or MB-222) resulted in enhanced lung specificity of the LNP, as evaluated by mRNA expression analysis in-vivo. Exemplary LNP compositions of the invention showed high lung specificity resulting in significantly enhanced expression of the encapsulated polynucleic acid (mRNA F-LUC) in the lung, as compared to a commercial control (FIG. 1). Furthermore, a specific LNP compositions of the invention (FMB-1143) comprising between about 15 and about 30% of the ionizable lipid exhibited superior lung specificity (see FIG. 1).

### Preparation of FMB-745

40% of DOTAP, 22.5% of cholesterol, 2.5% DMG-PEG2000 and 35% of a commercial ionizable lipid (Dlin-MC3-DMA) were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into mRNA firefly luciferase (F-LUC) containing citrate buffer under constant mixing conditions. pH of the mixture was then elevated using PBS dilution and residual EtOH was removed prior to injection using dialysis prepared by the inventors have been characterized by an average particle size ranging between about 60 and about 130 nm.

### Preparation of FMB-748

40% of DOTAP, 22.5% of cholesterol, 2.5% DMG PEG2000 and 35% of ionizable MB-222 were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into mRNA F-LUC containing citrate buffer under constant mixing conditions. pH of the mixture was then elevated using PBS dilution and residual EtOH was removed prior to injection using dialysis prepared by the inventors have been characterized by an average particle size ranging between about 60 and about 130 nm; N:P ratio of 8-9, and by an average Z of about +4 mV.

### Preparation of FMB-1143

40% of DOTAP, 38.5% of cholesterol, 1.5% DMG-PEG2000 and 20% of ionizable MB-212 were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into the mRNA F-LUC containing citrate buffer under constant mixing conditions. pH of the mixture was then elevated using PBS dilution and residual EtOH was removed prior to injection using dialysis prepared by the inventors have been characterized by an average particle size ranging between about 60 and about 130 nm; N:P ratio of 4-5, and by an average Z of about +3 mV.

The inventors determined the expression distribution of the exemplary LNPs in vivo by assessing expression of the encapsulated mRNA (mRNA F-LUC). The LNPS of the invention encapsulating mRNA F-LUC have been injected intravenously into a BALB/c mice, at a dosage of 13 μg/mouse (0.52-0.365 mg/kg). In-vivo imaging was done by IVIS imaging. Ex-vivo tissue analysis was done for lung, heart, spleen, kidney, and liver using IVIS. Histology assessment was determined by H&E staining with pathologist report for toxicity. Histology assessment concluded normal morphology without any treatment related pathological changes.

The expression distribution profile presented in FIG. 1 showed more than 100 times greater Luciferase signal in the lung as compared to heart, liver, spleen and kidney, indicating superior lung specificity of the exemplary LNPs of the invention.

Additionally, a similar formulation (FMB-393) including a zwitterionic helper lipid (DOPE) and characterized by a negative Z potential (about −22 mV) exhibited mostly liver expression/accumulation (as opposed to cationic helper lipid, as well as a positive Z potential of the exemplary formulations of the invention).

FMB-393 has been prepared, as described above. The chemical composition of the FMB-393 is as disclosed in Table 1.

TABLE 1

| Ionizable lipid | Helper lipid | Structural lipid | PEG lipid | |
|---|---|---|---|---|
| [mol %] | [mol %] | [mol %] | [mol %] | N:P |
| MB-212 [50] | DOPE [10] | Cholesterol [38.5] | DMG-PEG | 8 |
| | | | 2000 [1.5] | |

FMB-393 has been injected intravenously into a BALB/c mice and the expression distribution profile in-vivo has been assessed, as disclosed above.

The expression distribution profile of FMB-393 is presented in Table 2.

| | Fluorescence intensity |
|---|---|
| Organ | (from luciferase expression) |
| Heart | 0.00E+00 |
| Lungs | 4.04E+06 |
| Spleen | 4.57E+07 |
| Kidneys | 4.78E+06 |
| Liver | 6.41E+08 |

As evident from Table 2, FMB-393 showed mostly liver accumulation with more than 100 times greater Luciferase signal in the liver, as compared to lungs.

The inventors determined the expression distribution of the exemplary LNPs in vivo by assessing expression of the encapsulated mRNA (mRNA mCherry). The LNPS of the invention encapsulating mRNA mCherry have been injected through intravenous infusion into Cynomolgus monkey, at a dosage of 0.6 mg/kg. Tissue samples were collected 24 hours post administration from the following tissues: lung, brain, liver, heart, spleen, kidney, testes, mesenteric lymph node. The samples were analyzed using immunohistochemistry for expressed mCherry signal, compared to same samples collected from control animal injected with PBS/sucrose. The tissue analysis revealed intermediate to strong positive mCherry staining within the lung tissue sample.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A lipid nanoparticle comprising a compound, a salt of said compound or both, wherein the compound is represented by Formula 1: 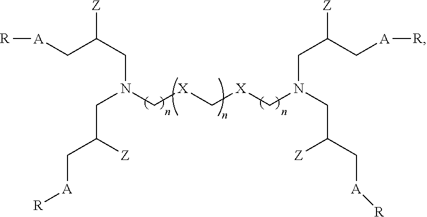
wherein:
each X independently represents a —O—, —S—, CH2, or X is absent;
each Z independently represents —OH or —SH;
each A independently represents O or S;
each n is independently between 0 and 5, and at least one n is not 0;
each R independently is H, or comprises an optionally substituted C5-C30 alkyl;
wherein said lipid nanoparticle further comprising an active agent, a helper lipid, a structural lipid and a modified lipid wherein:
the helper lipid is a cationic lipid;
a ratio of said compound relative to a total lipid content of said lipid nanoparticle is between 10 and 50 mol %; and
a ratio of said structural lipid relative to said total lipid content of said lipid nanoparticle is between 5 and 50 mol %; and wherein said lipid nanoparticle is characterized by an average zeta potential in a range between −5 and +40 mV at a pH between 6 and 8.

2. The lipid nanoparticle of claim 1, wherein a molar ratio of said helper lipid and said modified lipid is between 1:0.2 and 1:0.01.

3. The lipid nanoparticle of claim 1, wherein a molar ratio of said compound and said helper lipid is between 0.2:1 and 5:1.

4. The lipid nanoparticle of claim 1, wherein a molar ratio of said structural lipid and said modified lipid is between 1:0.01 and 1:0.2.

5. The lipid nanoparticle of claim 1, wherein said active agent comprises a polynucleic acid; and a ratio between N:P within said LNP is between 1 and 20.

6. The lipid nanoparticle of claim 1, wherein a weight ratio between (i) the total amount of said compound, said helper lipid, said structural lipid and said modified lipid, and (ii) the polynucleic acid within said lipid nanoparticle is between 0.001:1 and 10:1

7. The lipid nanoparticle of claim 1, wherein a size distribution of said lipid nanoparticles is in a range between 50 and 500 nm; and wherein the cationic lipid is selected from DOTAP, DDAB, 18:1 EPC(1,2-dioleoyl-sn-glycero-3-ethylphosphocholine), and 18:0 EPC (1,2-distearoyl-sn-glycero-3-ethylphosphocholine), including any salt and any combination thereof.

8. The lipid nanoparticle of claim 5, wherein:
the lipid nanoparticle is characterized by an average zeta potential between 0 and +5 mV, at a pH between 6 and 8;
the ratio between N:P is between 3 and 9;
said structural lipid is cholesterol;
said modified lipid is a PEG-lipid;
a ratio of said helper lipid relative to said total lipid content of said lipid nanoparticle is between 35 and 45 mol %; and
a ratio of the PEG-lipid relative to said total lipid content of said lipid nanoparticle is between 0.1 and 3 mol %.

9. The lipid nanoparticle of claim 1, wherein said lipid nanoparticles are characterized by pKa between 5 and 9; and wherein the compound comprises any one of MB-212 and MB 222:

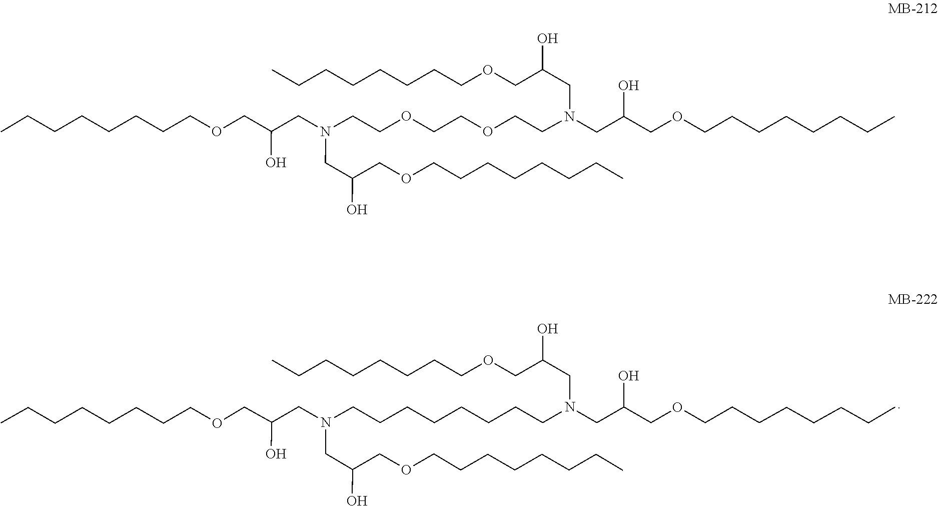

10. The lipid nanoparticle of claim 1, wherein upon administration to a subject, said LNP is characterized by at least 10 times higher expression within a lung, as compared to any one of liver, spleen, and kidney of said subject.

11. The lipid nanoparticle of claim 10, wherein said ratio between N:P is about 5; wherein the cationic lipid is DOTAP; and wherein a ratio of said compound relative to a total lipid content of said lipid nanoparticle is between about 15 and about 25 mol %.

12. A pharmaceutical composition comprises a plurality of the lipid nanoparticles of claim 1 and a pharmaceutically acceptable carrier.

13. The pharmaceutical composition of claim 12, comprising a therapeutically effective amount of said active agent.

14. (canceled)

15. (canceled)

16. A method for delivering an active agent to a lung tissue of a subject, the method comprising administering to said subject a therapeutically effective amount of the pharmaceutical composition of claim 12, thereby delivering the active agent to said lung tissue.

17. The method of claim 16, wherein said therapeutically effective amount is between 0.01 and 5 mg/kg per day.

18. The method of claim 16, wherein said administering is via intravenous, intratracheal, intranasal administration or via inhalation.
